(19) United States
(12) Patent Application Publication
(10) Pub. No.: US 2006/0234375 A1
(43) Pub. Date: Oct. 19, 2006

(54) Use of human stem cells and/or factors they produce to promote adult mammalian cardiac repair through cardiomyocyte cell division

(76) Inventors: Sergey V. Doronin, Stony Brook, NY (US); Ira S. Cohen, Stony Brook, NY (US); Peter R. Brink, Setauket, NY (US); Glenn Gaudette, Holden, MA (US); Michael R. Rosen, New York, NY (US); Richard B. Robinson, Cresskill, NJ (US)

Correspondence Address: COOPER & DUNHAM, LLP, 1185 AVENUE OF THE AMERICAS, NEW YORK, NY, 10036, US

(21) Appl. No.: 11/240,948

(22) Filed: Sep. 29, 2005

Related U.S. Application Data
(60) Provisional application No. 60/614,856, filed on Sep. 30, 2004.

(51) Int. Cl. C12N 5/08; C12N 5/00; A01N 63/00

(52) U.S. Cl. 435/366; 424/93.7; 435/375

(57) ABSTRACT

A method for treating a subject afflicted with a cardiac disorder, in vivo, comprising (i) producing a solution comprising media conditioned from the culture of cells, in vitro, and (ii) administering the solution of step (i) to the subject, thereby treating the cardiac disorder in the subject. Methods for determining whether an agent stimulates or inhibits myocyte proliferation.

### STATEMENT REGARDING FEDERALLY SPONSORED RESEARCH OR DEVELOPMENT

This research was supported by NIH HL20558 grant. The United States Government may have rights in this invention.

### BACKGROUND OF THE INVENTION

Throughout this application, various publications are referenced to by numbers. Full citations may be found at the end of the specification immediately preceding the claims. The disclosures of these publications in the entireties are hereby incorporated by reference into this application in order to more fully describe the state of the art to those skilled therein as of the date of the invention described and claimed herein.

Myocardial infarction leads to irreparable damage of the myocardium. Because of the lack of functional repair following infarction, and the low rate of self renewal there is the common belief that the mammalian heart is incapable of regeneration.

Following myocardial infarction, the heart does not reconstitute lost cardiomyocytes and the damaged tissue is eventually replaced by scar. This, however, does not rule out that regeneration of mammalian heart might occur under circumstances different from those of infarcted heart. For instance, zebrafish (1) or amphibians (2,3) reconstitute amputated parts of the heart and, in amphibians, heart regeneration occurs as a result of mitotic expansion of cardiomyocytes (2-4).

### SUMMARY OF THE INVENTION

The mammalian heart has an untapped potential to restore lost myocardium. To demonstrate this, a full thickness portion of the canine right ventricle was replaced with a material made of natural extracellular matrix. Myocardium was partially regenerated eight weeks later that produced significant regional mechanical work. This regeneration was accompanied by propagation of c-kit positive cells in the implant at early stages of the regeneration process, and was later associated with a mitotically expanding population of cardiomyocytes. It appeared that the interaction of stem cells with cardiomyocytes induced the later to enter the cell cycle. This process was reconstituted in vitro by co-culturing cardiomyocytes with human mesenchymal stem cells or treating cardiomyocytes with conditioned media from the human mesenchymal stem cells and observing cardiomyocytes proliferation. Given the proper environment, the mammalian heart can regenerate lost myocardium.

According to one aspect of the invention, a method for regenerating myocardium in a mammal is provided, comprising delivering cells to the myocardium that induce native myocytes to enter the cell cycle.

According to another aspect of the invention, a method for regenerating myocardium in a mammal is provided, comprising attracting native stem cells to the myocardium that induce native myocytes to enter the cell cycle.

According to another aspect of the invention, a solution that induces myocyte proliferation is provided, comprising at least one of (i) media conditioned by stem cells and (ii) media conditioned by myocytes and stem cells when they are co-cultured together.

According to another aspect of the invention, a method for producing a solution capable of inducing myocyte proliferation is provided, comprising delivering in vivo, media conditioned by stem cells.

According to another aspect of the invention, a method for producing a solution capable of inducing myocyte proliferation is provided, comprising delivering in vivo, media conditioned by stem cells co-cultured with myocytes.

According to another aspect of the invention, a method for treating a subject afflicted with a cardiac disorder, in vivo, is provided, comprising (i) producing a solution capable of inducing myocyte proliferation and (ii) administering the solution of step (i) to the subject, thereby treating the cardiac disorder in the subject.

According to another aspect of the invention, a method for treating a subject afflicted with a cardiac disorder, in vivo, is provided, comprising (i) producing a solution comprising media conditioned from the culture of cells, in vitro, and (ii) administering the solution of step (i) to the subject, thereby treating the cardiac disorder in the subject.

According to another aspect of the invention, a method for treating a subject afflicted with a cardiac disorder, in vivo, is provided, comprising (i) producing a solution comprising media conditioned from the co-culturing, in vitro, of cells and myocytes and (ii) administering the solution of step (i) to the subject, thereby treating the cardiac disorder in the subject.

According to another aspect of the invention, a method of effecting delivery of stem cells to an afflicted area of a heart is provided, comprising (i) excising a portion of the afflicted area and (ii) replacing the excised portion of step (i) with extracellular matrix which attracts stem cells, thereby causing stem cells to be delivered to the afflicted area of the heart.

According to another aspect of the invention, a method of determining whether an agent stimulates myocyte proliferation is provided, comprising (i) culturing, in vitro, cells and myocytes separately in the absence of the agent; (ii) exchanging the myocyte media with that from the cells; (iii) measuring the amount of myocyte cell division after step (ii); (iv) repeating steps (i) and (ii) by adding the agent to the media conditioned by the cells and exchanged for the myocyte media; (v) measuring the amount of myocyte cell division after step (iv); and (vi) comparing the measurements of step (iii) and step (v), whereby the amount of myocyte cell division as measured in step (v) being greater than the amount of myocyte cell division as measured in step (iii) indicates that the presence of the agent stimulates myocyte proliferation.

According to another aspect of the invention, a method of determining whether an agent stimulates myocyte proliferation is provided, comprising: (i) co-culturing, in vitro, cells and myocytes in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being greater than the amount of myocyte cell division as measured in step (ii) indicates that the presence of the agent stimulates myocyte proliferation.

According to another aspect of the invention, a method of determining whether an agent inhibits myocyte proliferation is provided, comprising: (i) culturing, in vitro, cells and myocytes separately; (ii) exchanging the media from the cells with the media of the myocytes in the absence of the agent; (iii) measuring the amount of myocyte cell division after step (i); (iv) repeating steps (i) and (ii) in the presence of the agent; (v) measuring the amount of myocyte cell division after step (iv); and (vi) comparing the measurements of step (iii) and step (v), whereby the amount of myocyte cell division as measured in step (v) being less than the amount of myocyte cell division as measured in step (iii) indicates that the presence of the agent inhibits myocyte proliferation.

According to another aspect of the invention, a method of determining whether an agent inhibits myocyte proliferation is provided, comprising: (i) co-culturing, in vitro, cells and myocytes, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being less than the amount of myocyte cell division as measured in step (ii) indicates that the presence of the agent inhibits myocyte proliferation.

According to another aspect of the invention, a method of determining whether an agent stimulates myocyte proliferation is provided, comprising: (i) delivering, in vivo, media conditioned by culture of cells, in vitro, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being greater than the amount of myocyte cell division as measured in step (ii) indicates that the presence of the agent stimulates myocyte proliferation.

According to another aspect of the invention, a method of determining whether an agent inhibits myocyte proliferation is provided, comprising: (i) co-incubating, in vivo, media conditioned by cells and media conditioned by myocytes, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being less than the amount of myocyte cell division as measured in step (ii) indicates that the presence of the agent inhibits myocyte proliferation.

According to another aspect of the invention, a method of determining whether an agent stimulates myocyte proliferation is provided, comprising: (i) delivering, in vivo, media conditioned by co-culture of cells and myocytes in vitro, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being greater than the amount of myocyte cell division as measured in step (ii) indicates that the presence of the agent stimulates myocyte proliferation.

According to another aspect of the invention, a method of determining whether an agent inhibits myocyte proliferation is provided, comprising: (i) co-incubating, in vivo, media conditioned by cells and media conditioned by myocytes, in the absence of the agent; (ii) measuring the amount of myocyte cell division after step (i); (iii) repeating step (i) in the presence of the agent; (iv) measuring the amount of myocyte cell division after step (iii), and (v) comparing the measurements of step (ii) and step (iv), whereby the amount of myocyte-cell division as measured in step (iv) being less than the amount of myocyte cell division as measured in step (ii) indicates that the presence of the agent inhibits myocyte proliferation.

According to another aspect of the invention, a method for stimulating cardiomyocytes to enter a cell cycle is provided, comprising co-culturing cells and cardiomyocytes.

According to another aspect of the invention, a method for stimulating cardiomyocytes to enter a cell cycle is provided, comprising culturing cardiomyocytes in media conditioned by cells.

According to another aspect of the invention, a method for stimulating cardiomyocytes to enter a cell cycle is provided, comprising culturing cardiomyocytes in media conditioned by cardiomyocytes and cells co-cultured.

According to another aspect of the invention, a method for determining whether a certain factor affects cardiomyocyte proliferation is provided, comprising: stimulating cardiomyocytes to enter a cell cycle under a certain set of conditions; determining the extent of cardiomyocyte proliferation according to step (a); stimulating cardiomyocytes to enter a cell cycle under the certain conditions of step (a), but changing at least one factor of the conditions; determining the extent of cardiomyocyte proliferation according to step (c); and comparing the results obtained from steps (b) and (d) to determine whether the factor affected cardiomyocyte proliferation.

### DESCRIPTION OF THE DRAWINGS

FIG. 1. Cardiac regeneration with ECM matrix. FIG. 1A shows regional stroke work of the myocardium distant from the site of surgery (Baseline), eight week old ECM implant region (ECM, *=p<0.05 from Baseline) and Dacron implant region (Dacron, #=p<0.05 from ECM). FIG. 1B shows staining of the eight weeks old ECM implant region for α-sarcomeric actinin (a cardiomyocyte marker; FITC, green). Nuclei were counterstained with DAPI (blue). Schematic location of implant—myocardium border (dashed green line), the border of regenerated myocardium (solid red line), area A of the adjacent host myocardium, area B of the internal part of the implant, area C of the tip of the regeneration cone and non regenerated area D of the implant overlaid on haematoxylin and eosin staining of eight week old ECM implant region.

FIG. 2. Stem cells accumulation in Dacron and ECM implants. Two weeks old Dacron and ECM implants were stained for α-sarcomeric actinin (TRITC, red) and c-kit (FITC, green). Nuclei were counterstained with DAPI (blue). FIG. 2A shows the border area of the Dacron implant with the host myocardium. FIG. 2B shows the border area of the ECM implant with the host myocardium. FIG. 2C shows internal area of ECM implant.

FIG. 3. Expression of cell division markers cyclin D1, Ki-67 and Wnt-5A in regenerated myocardium. Eight week old ECM implants were stained for cyclin D1 and Ki-67. Cardiomyocytes were visualised by staining for α-sarcomeric actinin (green) Nuclei were counterstained with DAPI (blue). FIG. 3A, shows cyclin D1 staining (red) of the epicardial surface of the implant. The layer of cyclin D1 positive cells is located under the surface layer of cyclin D1 negative cells. FIG. 3B shows the endocardial side of the implant with cardiomyocytes positive for cyclin D1. FIG. 3C shows nuclear localization of Ki-67 (red) in cardiomyocytes in the regenerating area. FIG. 3D shows the epicardial surface of the implant stained for Wnt-5A (green) and Ki-67 (red). Cells in the epicardial surface are Wint-5A positive and located above the layer of Ki-67 positive cells. FIG. 3E shows the endocardial side of the implant stained for cyclin D1 (red) and Wnt-5A (green). The endocardial surface of the implant is composed of Wnt-5A positive cells (blue arrow). Cyclin D1 positive cardiomyocytes (white arrow) are located above the Wnt-5A positive cell layer.

FIG. 4. Effects of human mesenchymal stem cells on cardiomyocytes in cell culture. Cardiomyocytes from canine hearts were co-cultured with human mesenchymal stem cells for 3-30 days in DMEM containing 5% of fetal bovine serum. Cells were labeled with BrdU and stained for cyclin D1 and Ki-67. Cardiomyocytes were visualized by staining for α-sarcomeric actinin. Nuclei and mitotic chromosomes were counterstained with DAPI (blue). FIG. 4A shows cardiomyocytes maintained in the absence of hMSCs for three days and stained for cyclin D1 (red) and α-sarcomeric actinin (green). FIG. 4B shows cyclin D1 expression (red) that was induced in cardiomyocytes after three days of co-culturing with hMSCs. Cardiomyocytes after five days in cell culture with hMSCs are shown on FIG. C and D. FIG. 4C shows a myocyte in anaphase stained for cyclin D1 (red) and α-sarcomeric actinin (green). FIG. 4D shows Ki-67 positive cardiomyocytes in the intermediate phase of the cell cycle (yellow arrow) and mitotically inactive (Ki-67 negative) cardiomyocytes (white arrow). FIG. 4E shows a ten day old colony of cardiomyocytes labelled with BrdU (green). FIG. 4F shows two week old colonies of cardiomyocytes (white arrows) which are often interconnected by spontaneously contracting myocytes (blue arrow). FIG. 4G shows a thirty day old colony of cardiomyocytes that were viable and cyclin D1 positive. FIG. 4H shows two fourteen day old colonies of cardiomyocytes. Cardiomyocytes were stimulated to proliferate by media conditioned by human mesenchymal stem cells but were not cultured with them. Cardiomyocyte colonies (white arrows) formed on the surface of cardiac fibroblasts which were also present in the initial myocyte preparation. The yellow arrow in the insert points to a mitotically silent cardiomyocyte.

### DESCRIPTION OF THE INVENTION

According to one aspect of the invention, a method for regenerating myocardium in a mammal is provided, comprising: delivering cells to the myocardium that induce native myocytes to enter the cell cycle.

The cells may be stem cells. The stem cells may be human stem cells. The mammal may be a human. The cells may be progenitor cells. The human stem cells may be human mesenchymal stem cells. The human stem cells may be human hematopoietic stem cells. The human stem cells may be human endothelial stem cells. The human stem cells may be human embryonic stem cells. The cells may be delivered via a scaffold. The cells may be delivered via a synthetic scaffold. The cells may be delivered via a biological scaffold. The cells may be delivered via an extracellular matrix scaffold. The cells may be delivered via an injection into the blood stream. The cells may be delivered via an injection into a coronary artery. The cells may be delivered via an injection into a coronary vein. The cells may be delivered via an injection into the myocardium. The cells may be delivered via an injection into the pericardial space.

According to another aspect of the invention, a method for regenerating myocardium in a mammal is provided, comprising attracting native stem cells to the myocardium that induce native myocytes to enter the cell cycle.

The native stem cells may be attracted to the myocardium by (i) excising a portion of the myocardium and (ii) replacing the excised portion with an extracellular matrix. The stem cells may be human stem cells. The mammal may be a human. The stem cells may be progenitor cells. The human stem cells may be human mesenchymal stem cells. The human stem cells may be human hematopoietic stem cells. The human stem cells may be human endothelial stem cells. The human stem cells may be human embryonic stem cells. The stem cells may be delivered via a scaffold. The stem cells may be delivered via a synthetic scaffold. The stem cells may be delivered via a biological scaffold. The stem cells may be delivered via an extracellular matrix scaffold. The stem cells may be delivered via an injection into the blood stream. The stem cells may be delivered via an injection into a coronary artery. The stem cells may be delivered via an injection into a coronary vein. The stem cells may be delivered via an injection into the myocardium. The stem cells may be delivered via an injection into the pericardial space.

According to another aspect of the invention, a solution that induces myocyte proliferation is provided, comprising at least one of (i) media conditioned by stem cells and (ii) media conditioned by myocytes and stem cells when they are co-cultured together.

The media conditioned by the stem cells and the media conditioned by the coculturing of the stem cells and myocytes may be mixed together. The media conditioned by stem cells may be used to incubate myocytes. The solution may further comprise Wnt 5a. The solution may further comprise metalloproteases (MMPs). The solution may further comprise insulin-like growth factor. The solution may further comprise platelet derived growth factor. The solution may further comprise brain derived neurotrophic factor.

According to another aspect of the invention, a method for producing a solution capable of inducing myocyte proliferation is provided, comprising delivering in vivo, media conditioned by stem cells.

According to another aspect of the invention, a method for producing a solution capable of inducing myocyte proliferation is provided, comprising delivering in vivo, media conditioned by stem cells co-cultured with myocytes.

According to another aspect of the invention, a method for treating a subject afflicted with a cardiac disorder, in vivo, is provided, comprising (i) producing a solution capable of inducing myocyte proliferation and (ii) administering the solution of step (i) to the subject, thereby treating the cardiac disorder in the subject.

According to another aspect of the invention, a method for treating a subject afflicted with a cardiac disorder, in vivo, is provided, comprising (i) producing a solution comprising media conditioned from the culture of cells, in vitro, and (ii) administering the solution of step (i) to the subject, thereby treating the cardiac disorder in the subject.

The cardiac disorder may be myocardial infarction. The cardiac disorder may be cardiomyopathy. The cardiac disorder may be congestive heart failure. The cardiac disorder may be ventricular septal defect. The cardiac disorder may be atrial septal defect. The cardiac disorder may be congenital heart defect. The cardiac disorder may be ventricular aneurysm. The cardiac disorder may be pediatric in origin. The cardiac disorder may require ventricular reconstruction. The cells may be human stem cells. The subject may be a human. The cells may be progenitor cells. The cells may be stem cells. The human stem cells may be human mesenchymal stem cells. The human stem cells may be human hematopoietic stem cells. The human stem cells may be human endothelial stem cells. The human stem cells may be human embryonic stem cells. The solution may be administered via a scaffold. The solution may be administered via a synthetic scaffold. The solution may be administered via a biological scaffold. The solution may be administered via an extracellular matrix scaffold. The solution may be administered via an injection into the blood stream. The solution may be administered via an injection into a coronary artery. The solution may be administered via an injection into a coronary vein. The solution may be administered via an injection into the myocardium. The solution may be administered via an injection into the pericardial space.

According to another aspect of the invention, a method for treating a subject afflicted with a cardiac disorder, in vivo, is provided, comprising (i) producing a solution comprising media conditioned from the co-culturing, in vitro, of cells and myocytes and (ii) administering the solution of step (i) to the subject, thereby treating the cardiac disorder in the subject.

The cardiac disorder may be myocardial infarction. The cardiac disorder may be cardiomyopathy. The cardiac disorder may be congestive heart failure. The cardiac disorder may be ventricular septal defect. The cardiac disorder may be atrial septal defect. The cardiac disorder may be congenital heart defect. The cardiac disorder may be ventricular aneurysm. The cardiac disorder may be pediatric in origin. The cardiac disorder requires ventricular reconstruction. The cells may be stem cells. The stem cells may be human stem cells. The subject may be a human. The cells may be progenitor cells. The human stem cells may be human mesenchymal stem cells. The human stem cells may be human hematopoietic stem cells. The human stem cells may be human endothelial stem cells. The human stem cells may be human embryonic stem cells. The solution may be administered via a scaffold. The solution may be administered via a synthetic scaffold. The solution may be administered via a biological scaffold. The solution may be administered via an extracellular matrix scaffold. The solution may be administered via an injection into the blood stream. The solution may be administered via an injection into a coronary artery. The solution may be administered via an injection into a coronary vein. The solution may be administered via an injection into the myocardium. The solution may be administered via an injection into the pericardial space.

According to another aspect of the invention, a method of effecting delivery of stem cells to an afflicted area of a heart is provided, comprising (i) excising a portion of the afflicted area and (ii) replacing the excised portion of step (i) with extracellular matrix which attracts stem cells, thereby causing stem cells to be delivered to the afflicted area of the heart.

The excised portion may be about 10-15 mm in length and width.

According to another aspect of the invention, a method of determining whether an agent stimulates myocyte proliferation is provided, comprising (i) culturing, in vitro, cells and myocytes separately in the absence of the agent; (ii) exchanging the myocyte media with that from the cells; (iii) measuring the amount of myocyte cell division after step (ii); (iv) repeating steps (i) and (ii) by adding the agent to the media conditioned by the cells and exchanged for the myocyte media; (v) measuring the amount of myocyte cell division after step (iv); and (vi) comparing the measurements of step (iii) and step (v), whereby the amount of myocyte cell division as measured in step (v) being greater than the amount of myocyte cell division as measured in step (iii) indicates that the presence of the agent stimulates myocyte proliferation.

The agent may be a cell. The cells may be progenitor cells.

According to another aspect of the invention, a method of determining whether an agent stimulates myocyte proliferation is provided, comprising: (i) co-culturing, in vitro, cells and myocytes in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being greater than the amount of myocyte cell division as measured in step (ii) indicates that the presence of the agent stimulates myocyte proliferation.

The agent may be a cell. The cells may be progenitor cells.

According to another aspect of the invention, a method of determining whether an agent inhibits myocyte proliferation is provided, comprising: (i) culturing, in vitro, cells and myocytes separately; (ii) exchanging the media from the cells with the media of the myocytes in the absence of the agent; (iii) measuring the amount of myocyte cell division after step (i); (iv) repeating steps (i) and (ii) in the presence of the agent; (v) measuring the amount of myocyte cell division after step (iv); and (vi) comparing the measurements of step (iii) and step (v), whereby the amount of myocyte cell division as measured in step (v) being less than the amount of myocyte cell division as measured in step (iii) indicates that the presence of the agent inhibits myocyte proliferation.

The agent may be a cell. The cells may be progenitor cells.

According to another aspect of the invention, a method of determining whether an agent inhibits myocyte proliferation is provided, comprising: (i) co-culturing, in vitro, cells and myocytes, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being less than the amount of myocyte cell division as measured in step (ii) indicates that the presence of the agent inhibits myocyte proliferation.

The agent may be a cell. The cells may be progenitor cells.

According to another aspect of the invention, a method of determining whether an agent stimulates myocyte proliferation is provided, comprising: (i) delivering, in vivo, media conditioned by culture of cells, in vitro, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being greater than the amount of myocyte cell division as measured in step (ii) indicates that the presence of the agent stimulates myocyte proliferation.

The agent may be a cell. The cells may be progenitor cells.

According to another aspect of the invention, a method of determining whether an agent inhibits myocyte proliferation is provided, comprising: (i) co-incubating, in vivo, media conditioned by cells and media conditioned by myocytes, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being less than the amount of myocyte cell division as measured in step (ii) indicates that the presence of the agent inhibits myocyte proliferation.

The agent may be a cell. The cells may progenitor cells.

According to another aspect of the invention, a method of determining whether an agent stimulates myocyte proliferation is provided, comprising: (i) delivering, in vivo, media conditioned by co-culture of cells and myocytes in vitro, in the absence of the agent; (ii) measuring the amount of myocyte cell division after step (i); (iii) repeating step (i) in the presence of the agent; (iv) measuring the amount of myocyte cell division after step (iii); and (v) comparing the measurements of step (ii) and step (iv), whereby the amount of myocyte cell division as measured in step (iv) being greater than the amount of myocyte cell division as measured in step (ii) indicates that the presence of the agent stimulates myocyte proliferation.

The agent may be a cell. The cells may be progenitor cells.

According to another aspect of the invention, a method of determining whether an agent inhibits myocyte proliferation is provided, comprising: (i) co-incubating, in vivo, media conditioned by cells and media conditioned by myocytes, in the absence of the agent; (ii) measuring the amount of myocyte cell division after step (i); (iii) repeating step (i) in the presence of the agent; (iv) measuring the amount of myocyte cell division after step (iii), and (v) comparing the measurements of step (ii) and step (iv), whereby the amount of myocyte cell division as measured in step (iv) being less than the amount of myocyte cell division as measured in step (ii) indicates that the presence of the agent inhibits myocyte proliferation.

The agent may be a cell. The cells may be progenitor cells.

According to another aspect of the invention, a method for stimulating cardiomyocytes to enter a cell cycle is provided, comprising co-culturing cells and cardiomyocytes.

The cells may be progenitor cells.

According to another aspect of the invention, a method for stimulating cardiomyocytes to enter a cell cycle is provided, comprising culturing cardiomyocytes in media conditioned by cells.

The cells may be progenitor cells.

According to another aspect of the invention, a method for stimulating cardiomyocytes to enter a cell cycle is provided, comprising culturing cardiomyocytes in media conditioned by cardiomyocytes and cells co-cultured.

The cells may be progenitor cells.

According to another aspect of the invention, a method for determining whether a certain factor affects cardiomyocyte proliferation is provided, comprising: stimulating cardiomyocytes to enter a cell cycle under a certain set of conditions; determining the extent of cardiomyocyte proliferation according to step (a); stimulating cardiomyocytes to enter a cell cycle under the certain conditions of step (a), but changing at least one factor of the conditions; determining the extent of cardiomyocyte proliferation according to step (c); and comparing the results obtained from steps (b) and (d) to determine whether the factor affected cardiomyocyte proliferation.

The step (a) may comprise co-culturing stem cells with cardiomyocytes. The step (a) may comprise culturing cardiomyocytes in media conditioned by stem cells.

As used herein, the term “media” means a nutrient solution in which cells or organs are grown.

As used herein, the term “cell cycle” means a sequence of events between mitotic divisions of cells.

As used herein, the term “extracellular matrix” means a scaffold composed of organic matter.

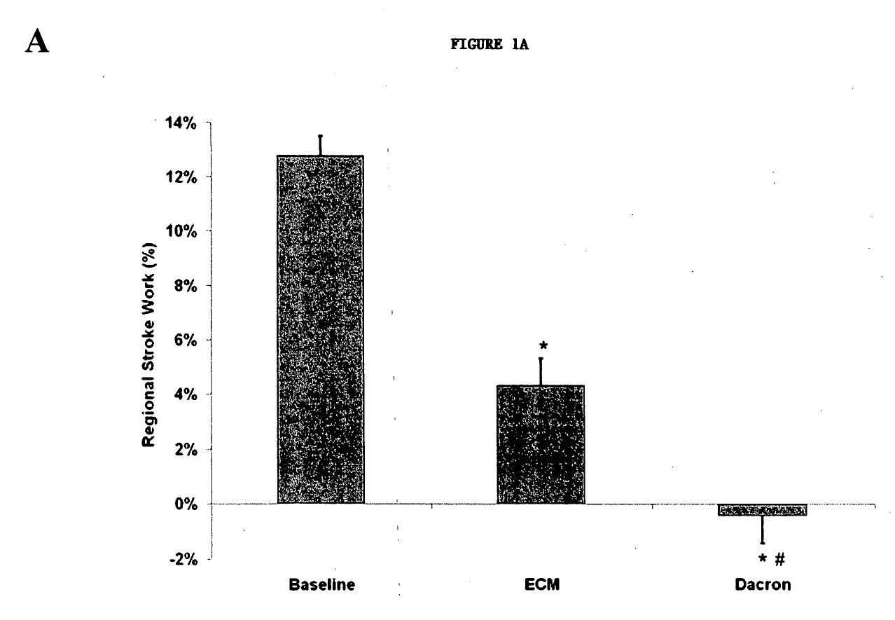

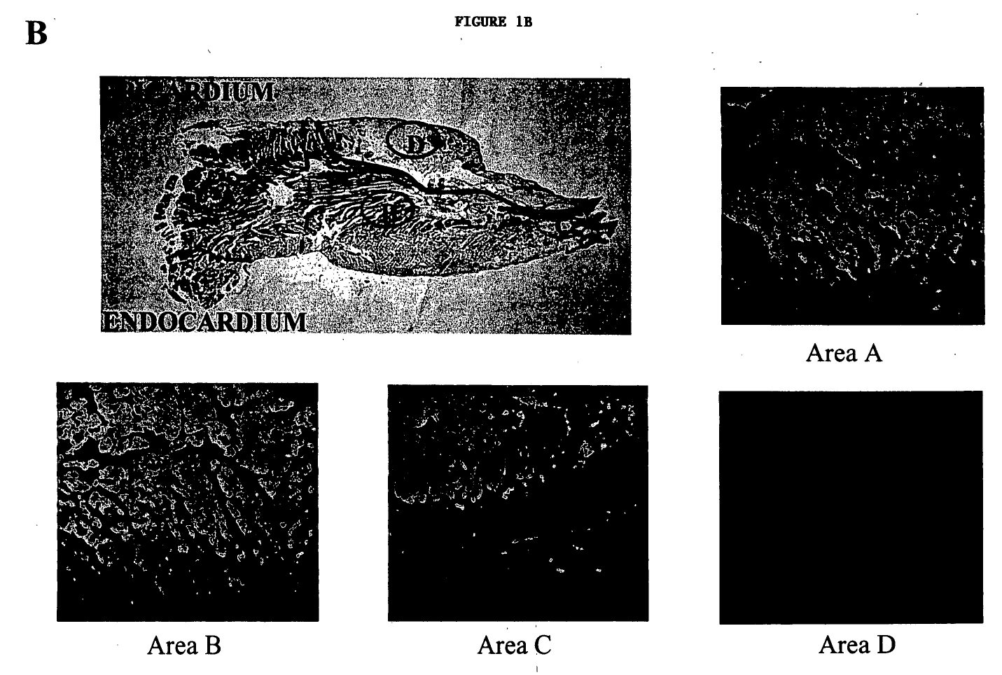

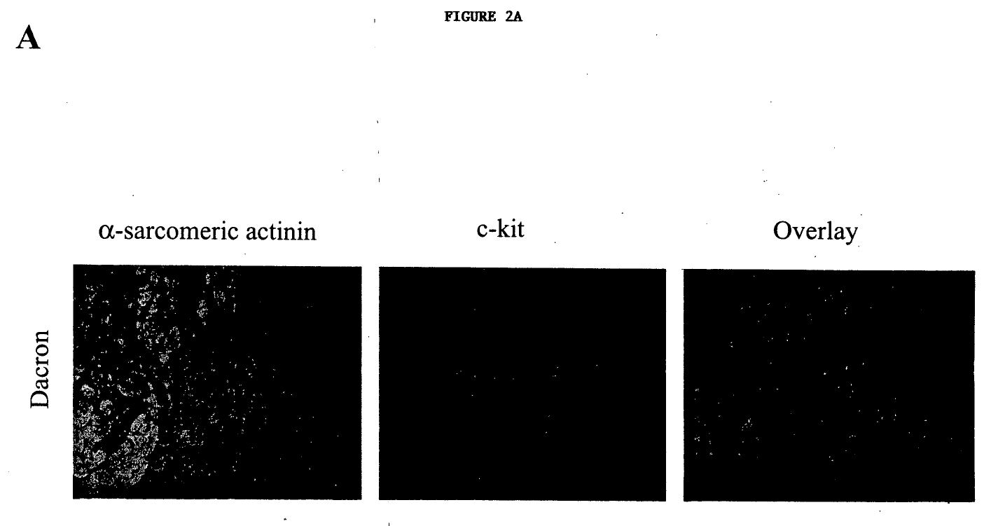

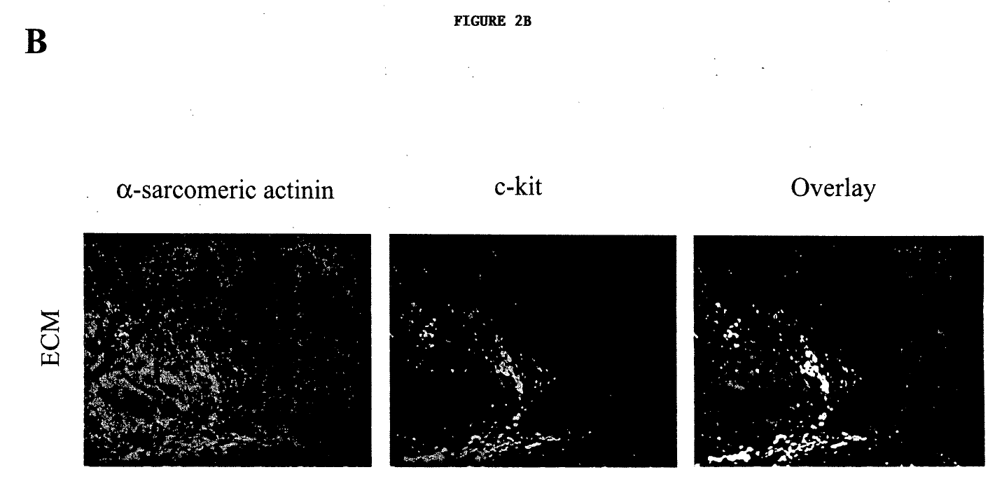

To investigate how generally healthy mammalian heart will respond to similar injuries a small (less than 5% of area) full thickness region of the canine right ventricle was excised and replaced with a patch made of either extracellular matrix (ECM) prepared from swine bladder (5) or Dacron synthetic material. Regional heart performance was assayed eight weeks after implantation as previously described (6,7). Non-implant regions of the heart (n=4) displayed a regional stroke work of 13±1% (normalized to developed pressure and end diastolic area), whereas Dacron implants (n=4) had 0±1% regional stroke work. Regional function was significantly better in the ECM implanted hearts (n=4) with a regional stroke work of 4±1% (FIG. 1A), suggesting that contractile function in the implant region was partially restored. Microscopic examination revealed that the myocardium was partially regenerated in the ECM implant (FIG. 1B). Cells with myocyte morphology that stained positively for α-sarcomeric actinin (α-SA) were located at the endocardial side of the ECM implant. In addition, a gradient of thickness from the periphery to the center of the patch was observed. No myocytes were found in Dacron implants. The absence of myocardial regeneration with the Dacron implants suggested that the extracellular matrix may play a crucial role in the process of regeneration.

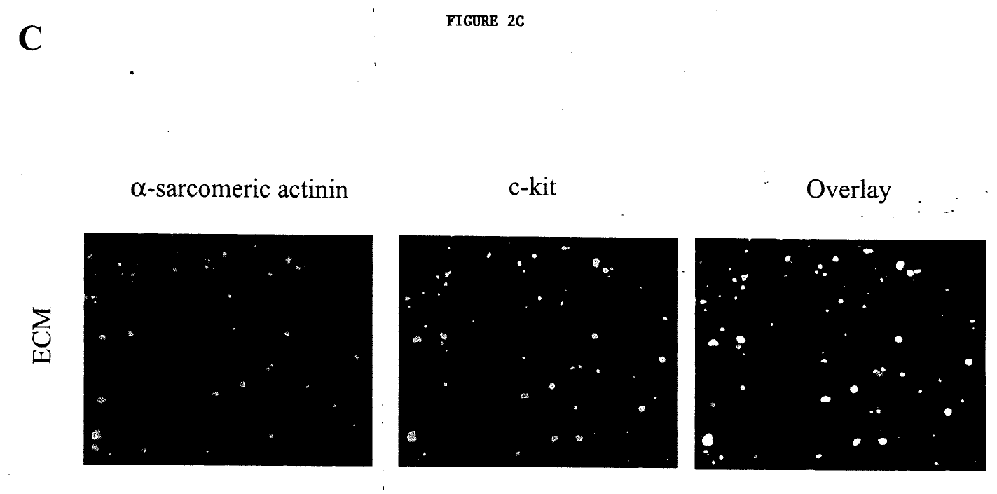

ECM assisted regeneration of canine myocardium demonstrates that like amphibians (2) or zebrafish (1) the mammalian heart can regenerate amputated myocardium. However, this process requires the presence of “healthy” extracellular matrix. To investigate the possible mechanisms of regeneration animals were assayed at two weeks post implantation of either Dacron or ECM for the presence of cells that are c-kit positive, as Lin⁻-c-kit⁺ stem cells have been reported to play a pivotal role in myocardial regeneration (8,9). ECM implants were found to be populated with c-kit⁺ cells gravitating to the mid-myocardium and endocardial regions of the implant. Adjacent host myocardium did not contain c-kit⁺ cells, suggesting that the stem cells may be derived from the blood stream. Proliferation of c-kit⁺ cells occurred at the border area of the implant and host myocardium (FIG. 2B). In contrast, the Dacron implants were depleted of c-kit⁺ cells (FIG. 2A), although, a small number could be found after thorough examination. Furthermore, in the ECM implants, many c-kit⁺ stem cells also stained positive for α-SA, independent of whether they made contact with the host myocardium (FIG. 2C). This suggests that direct contact with cardiomyocytes is not required for α-SA expression. Regeneration of myocardium in the presence of ECM correlates with the presence of c-kit⁺ cells. However, recent studies have shown that c-kit⁺ stem cells adopt mature hematopoetic fates in myocardium and do not transdifferentiate in cardiomyocytes (10,11). This suggest that differentiation of c-kit⁺ cells into myocytes might not be involved in the repair observed in the ECM implants, suggesting that myocardial regeneration occurs through a different mechanism.

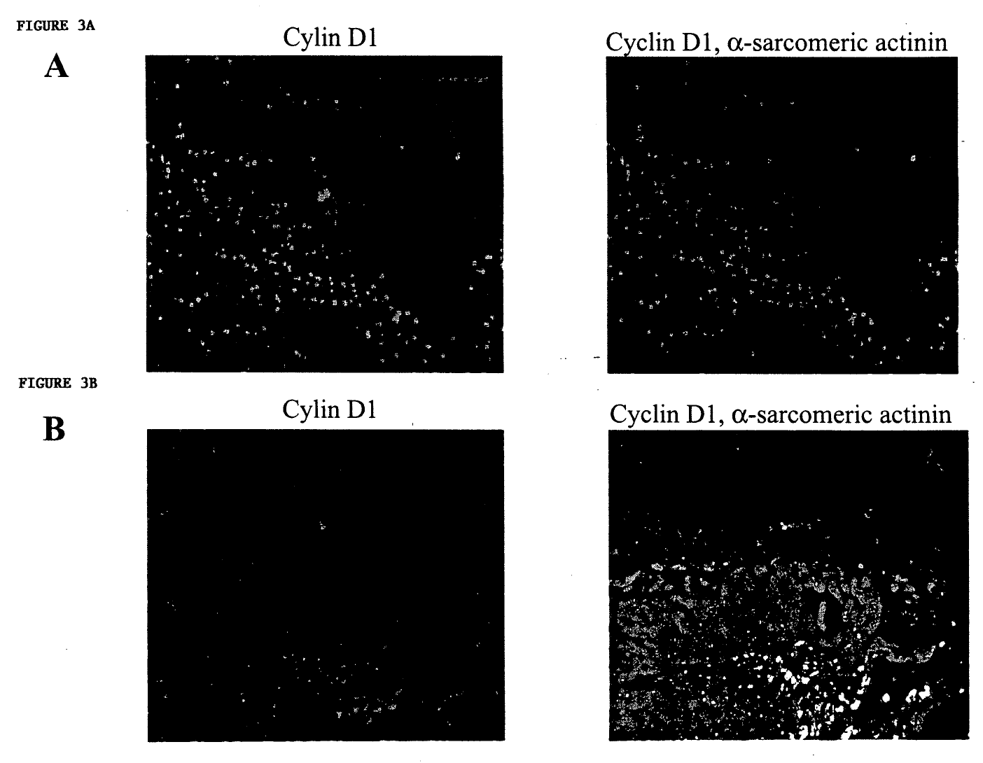

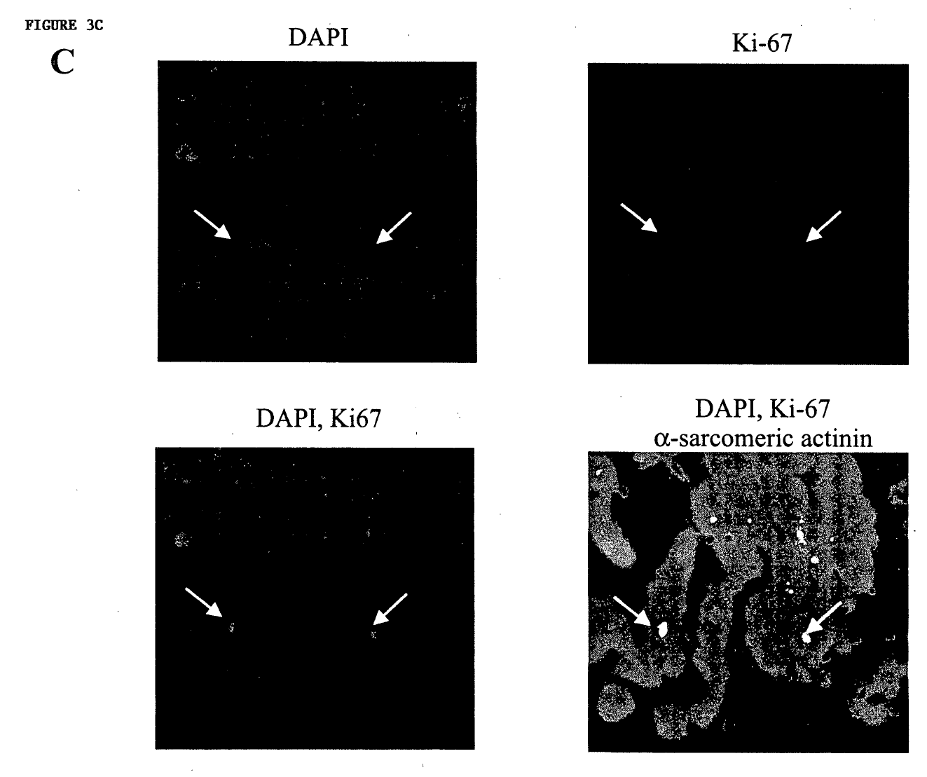

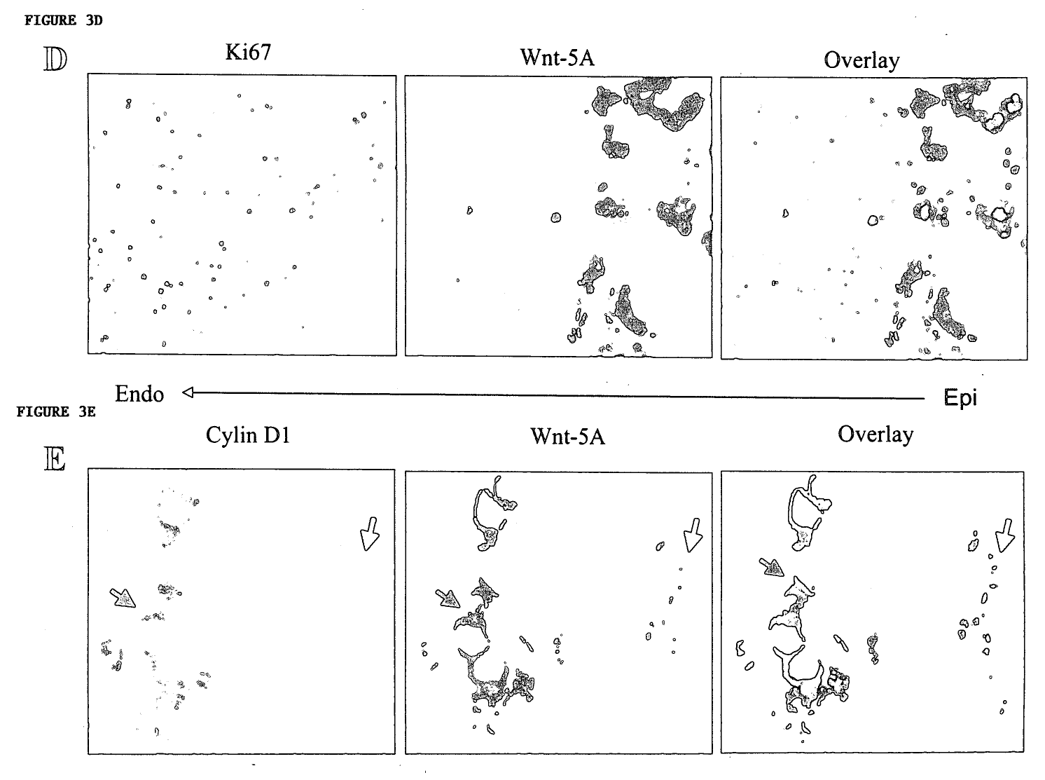

In considering alternatives, amphibian myocardium regenerates as a result of mitotic division of cardiomyocytes. To evaluate this mechanism of regeneration implants were examined for expression of two markers of cell division Ki-67 and cyclin D1. The two week ECM implants did not show improvement in regional contraction or myocardium reconstitution that is seen in the implant regions at eight weeks. Eight week ECM implants were essentially free of c-kit⁺ cells. Host myocardium adjacent to the implant at eight weeks (area A in FIG. 1B) did not show staining for Ki-67 or cyclin D1, demonstrating that this region was composed of mitotically silent cardiomyocytes. A similar result was obtained for the internal area of regenerated myocardium (area B in FIG. 1B). A layer of cells at the epicardial surface of the implant (area D in FIG. 1B) contained cyclin D1 positive cells (FIG. 3A). Cardiomyocytes at the tip of the regeneration cone (area C in FIG. 1B) were found to be cyclin D1 (FIG. 3B) and Ki-67 positive (FIG. 3C). These results show that mitotic expansion of myocytes might be part of the mechanism of myocardial regeneration in the ECM implants. This suggests that regeneration of the amputated mammalian heart might follow the mechanism of amphibian heart regeneration. The data obtained also correlate with heart regeneration in MRL mice (12). In the case of MRL mice, the mitotic index of myocytes (10-20%) during regeneration of cryogenically injured heart was close to that of amphibians (4). MRL mice regenerate wounds without forming scars, presumably due to an altered mechanism of ECM remodeling (12). ECM assisted myocardial regeneration shows that normal extracellular matrix attracts stem cells and later gives raise to a population of mitotically competent cardiomyocytes.

Cardiomyocytes leave the cell cycle shortly after birth and lose markers of cell division. Expression of Ki-67 and cyclin D1, the markers of mitotically competent cells, suggests that cardiomyocytes were exposed to stimulators of mitotic proliferation. To study the signals which support cellular proliferation, the distribution of Wnt-5A, a stimulator of cyclin D1 expression (13,14), was examined. Wnt-5A+ cells were located (FIG. 3D) above the layer of dividing cells (FIG. 3A) at the epicardial surface in the eight week ECM implant. A second layer of Wnt-5A⁺ cells was identified under the layer of proliferating cardiomyocytes (FIG. 3B) at the endocardial surface of the implant (FIG. 3E). Wnt-5A⁺ cells were not found in the myocardium of control dogs or in the area of host myocardium adjacent to the site of surgery. These data suggest that population of the ECM implant with stem cells establishes an environment resulting in the expression of signalling factors that are not present in the normal myocardium.

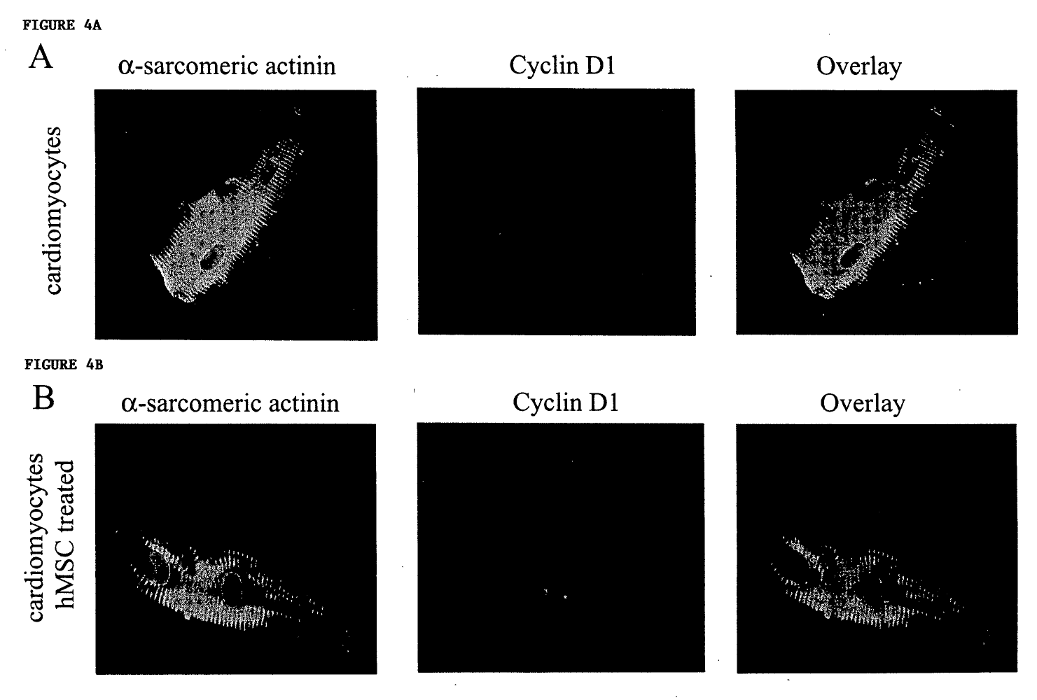

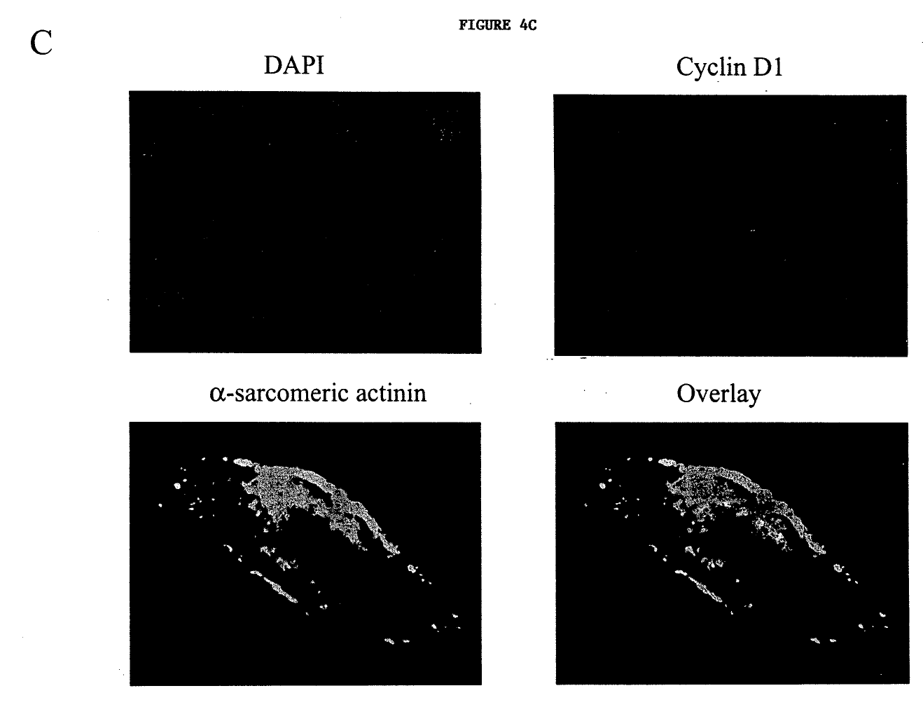

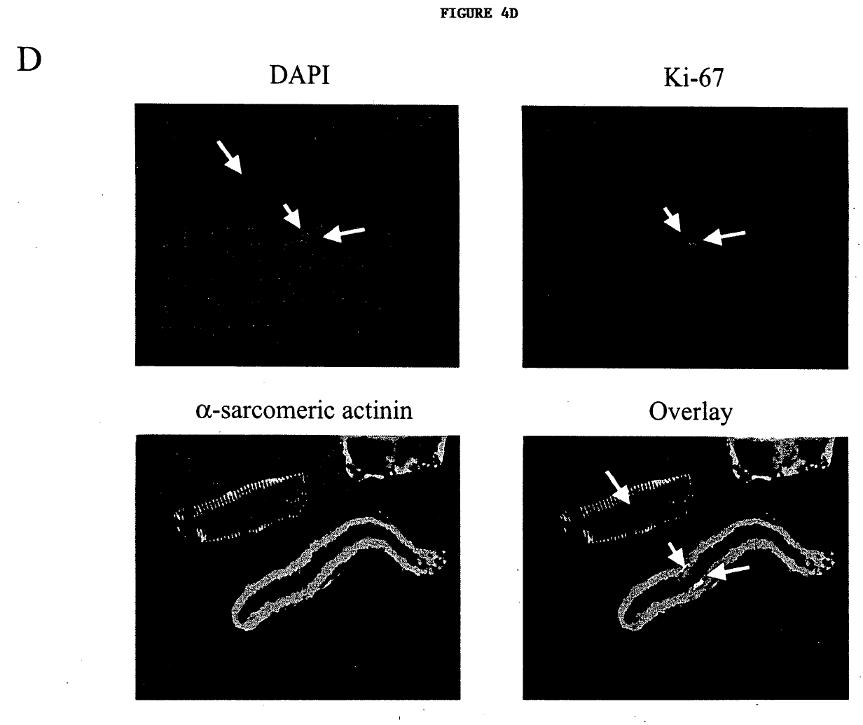

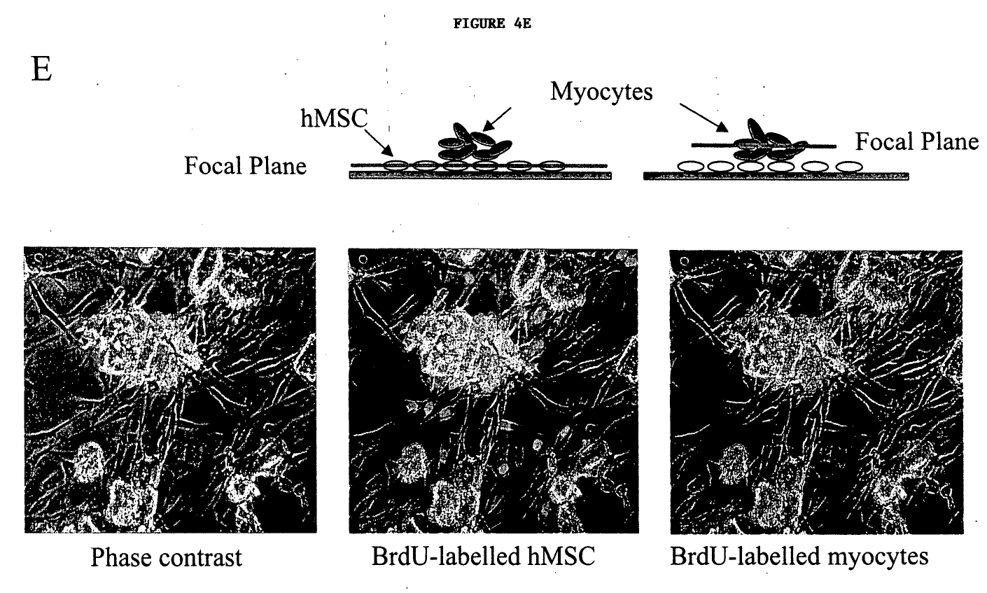

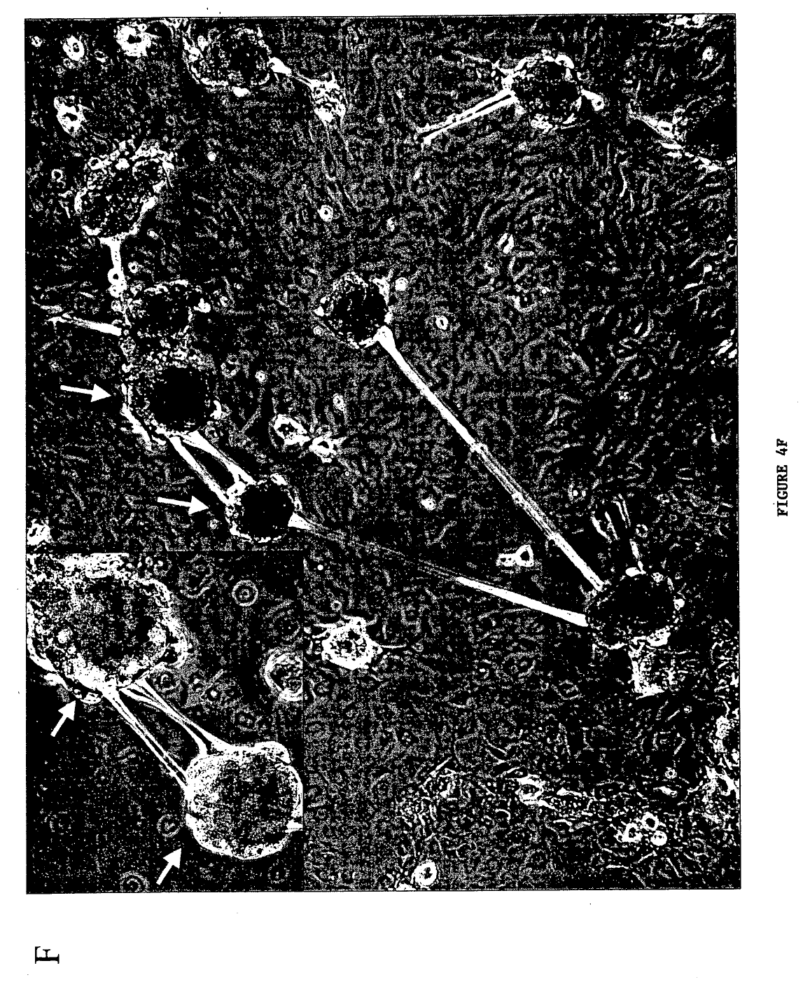

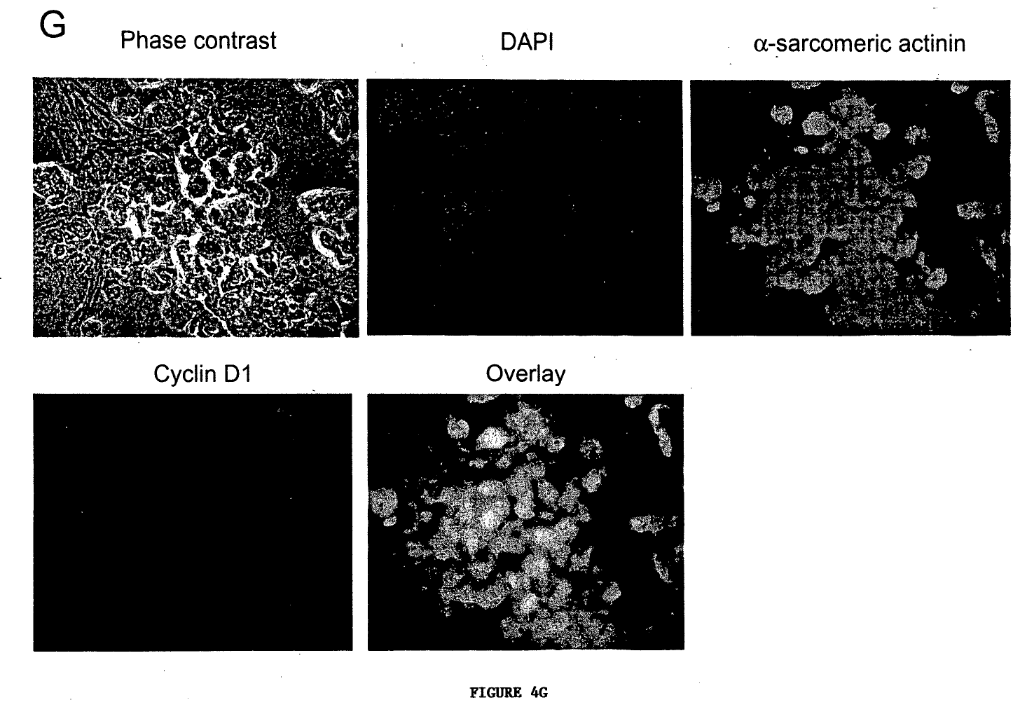

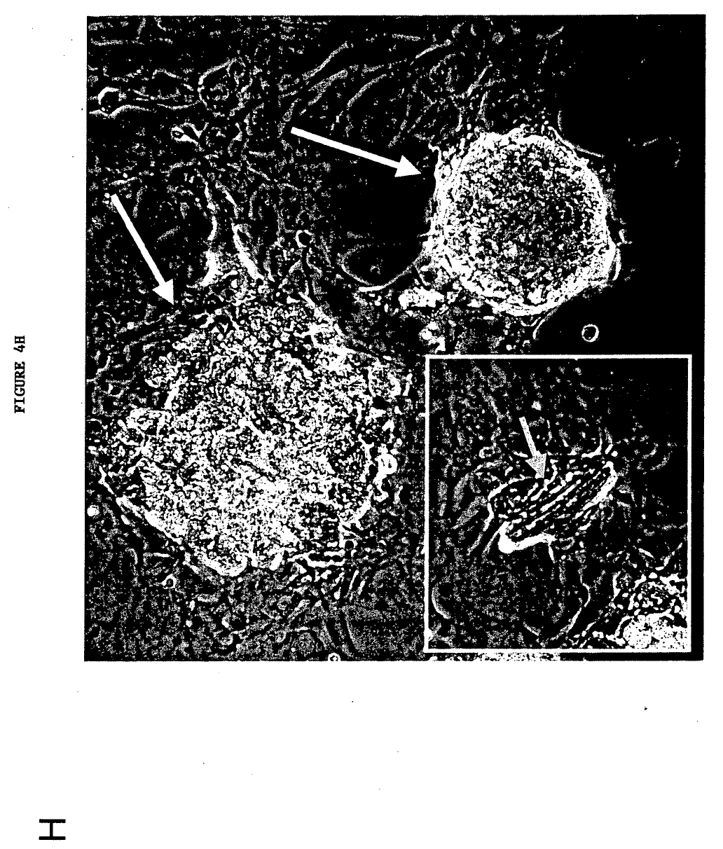

To mimic myocyte exposure to factors produced by stem cells in ECM implants, cardiomyocytes isolated from canine ventricle were co-cultured with human mesenchymal stem cells (hMSCs), without ECM. Mesenchymal stem cells have been shown to regenerate myocardium, although through a mechanism other than transdifferentiation into cardiomyocytes (15). These hMSCs produce a variety of signalling factors (16), including a set of Wnt proteins (16,17). After 3-4 days of co-culture, expression of cyclin D1 was detected in cardiomyocytes, whereas control cardiomyocytes, maintained in the absence of stem cells, remain cyclin D1 negative (FIG. 4A,B). Intermediates of cell division were detected after 4-5 days of co-culture with hMSCs among the cyclin D1 and Ki-67 positive cardiomyocytes (FIG. 4C,D). After ten days co-cultured with hMSCs, cardiomyocytes formed colonies that included cells stained positive for DNA synthesis with BrdU (FIG. 4E). Two week old colonies of cardiomyocytes were often interconnected by spontaneously contracting myocytes (FIG. 4F). During the next thirty days colonies of myocytes proliferated and formed conical structures on the surface of the hMSCs that included dozens of cyclin D1 and Ki-67 positive cardiomyocytes (FIG. 4G). These colonies were viable for at least three months.

The experiments discussed above with ECM implants demonstrate that the lack of regeneration in the mammalian heart is likely related to an unfavourable environment, rather than an innate inability of the mammalian myocardium to regenerate. Replacement of myocardium with normal extracellular matrix creates an environment that is favourable to myocardial regeneration. These favourable conditions are characterized by proliferation of c-kit⁺ stem cells that change the signalling pattern in the myocardium. Our in vitro model does not involve the use of ECM, thereby suggesting its importance in providing an environment for proliferating cells, rather than stimulating cells to proliferate. Our in vitro model further suggests that interaction of myocytes with stem cells can induce cardiomyocytes to enter the cell cycle, and it is this entry into the cell cycle that is likely to be an important part of stem cell assisted myocardium regeneration. In fact even conditioned media from the cultured hMSCs can induce myocyte proliferation (FIG. 4, panel H). This means that conditioned media independent of cells can in principle induce cardiac repair through myocyte proliferation.

Methods

Human mesenchymal stem cells were obtained from BioWhittaker/Cambrex Inc. Cyclin-D1, Ki-67 and c-kit antibodies were purchased from Santa Cruz Biotechnology Inc. Antibody for α-sarcomeric actinin was purchased from Sigma. Urinary bladder extracellular matrix membrane (ECM) was generously provided by Dr. Stephan Badylak (University of Pittsburgh).

To introduce an amputation wound in the canine heart a full thickness portion of myocardium of right ventricle approximately 15×10 mm was excised and replaced with membrane made of ECM or Dacron in adult mongrel dogs. All animal received humane care in accordance with the “Principles of Laboratory Animal Care” formulated by the National Society for Medical Research and the “Guide for the Care and Use of Laboratory Animals” of National Academy of Sciences (NIH publication No. 85-23) and treated according to protocol IACUC#20031326 approved by the Animal Care and Use Committee at SUNY Stony Brook.

For in vitro experiments canine ventricular cardiomyocytes were isolated in thyroid solution as described (18) supplied with 10 nM insulin and placed on poly-D-lysine—laminin coated 35 mm cell culture dishes or in Lab-Tek II CC2 chamber slides (BD Biosciencies). Myocytes were maintained in a humidified atmosphere of 5% CO₂ at 37° C. After 3-4 hours, thyroid solution was replaced with serum free DMEM media containing 10 nM insulin. After 9-12 hours, cardiomyocytes cells were washed twice with DMEM and supplied with hMSCs in DMEM containing 5% fetal bovine serum to produce 50% confluent monolayer of hMSCs. Media was changed once every four days.

Fluorescent images of formaldehyde fixed tissue slices or cells cultured in vitro were acquired with Carl Zeiss Axiovert 200M fluorescent microscope. Normanski DIC images were deconvoluted with AxioVision software package (Carl Zeiss).

### REFERENCES

1. Poss, K. D., Wilson, L. G. & Keating, M. T. Heart regeneration in zebrafish. Science 298, 2188-2190 (2002).
2. Rumyantsev, P. P. Post-injury DNA synthesis, mitosis and ultrastructural reorganization of adult frog cardiac myocytes. An electron microscopic-autoradiographic study. Z. Zellforsch. Mikrosk. Anat. 139, 431-450 (1973).
3. Flink, I. L. Cell cycle reentry of ventricular and atrial cardiomyocytes and cells within the cpicardium following amputation of the ventricular apex in the axolotl, Amblystoma mexicanum: confocal microscopic immunofluorescent image analysis of bromodeoxyuridine-labeled nuclei. Anat. Embryol. (Berl) 205, 235-244 (2002).
4. Borisov, A. B. Cellular and molecular basis of regeneration from invertebrates to humans. Wiley, New York (1998).
5. Badylak, S. F. The extracellular matrix as a scaffold for tissue reconstruction. Semin. Cell Dev. Biol. 13, 377-383 (2002).
6. Gaudette, G. R. et al. Determination of regional area stroke work with high spatial resolution in the heart. Cardiovascular Engineering: An International Journal 2, 129-137 (2002).
7. Gaudette, G. R., Todaro, J., Krukenkamp, I. B. & Chiang, F. P. Computer aided speckle interferometry: a technique for measuring deformation of the surface of the heart. Ann. Biomed. Eng 29, 775-780 (2001).
8. Beltrami, A. P. et al. Adult cardiac stem cells are multipotent and support myocardial regeneration. Cell 114, 763-776 (2003).
9. Orlic, D. et al. Bone marrow stem cells regenerate infracted myocardium. Pediatr. Transplant. 7 Suppl 3, 86-88 (2003).
10. Balsam, L. B. et al. Haematopoietic stem cells adopt mature haematopoietic fates ischaemic myocardium. Nature 428, 668-673 (2004).
11. Murry, C. E. et al. Haematopoietic stem cells do not transdifferentiate into cardiac myocytes in myocardial infarcts. Nature 428, 664-668 (2004).
12. Leferovich, J. M. et al. Heart regeneration in adult MRL mice. Proc. Natl. Acad. Sci. U.S. A 98, 9830-9835 (2001).
13. Shtutman, M. et al. The cyclin D1 gene is a target of the beta-catenin/LEF-1 pathway. Proc. Natl. Acad. Sci. U.S. A 96, 5522-5527 (1999).
14. Tetsu, O. & McCormick, F. Beta-catenin regulates expression of cyclin D1 in colon carcinoma cells. Nature 398, 442-426 (1999).
15. Rodic, N., Rutenberg, M. S. & Terada, N. Cell fusion and reprogramming: resolving our transdifferences. Trends Mol. Med 10, 93-96 (2004).
16. Doi, M., Nagano, A. & Nakamura, Y. Genome-wide Screening by cDNA Microarray of Genes Associated with Matrix Mineralization by Human Mesenchymal Stem Cells in Vitro. Biochemical and Biophysical Research Communications 290, 381-390 (2002).
17. Gregory, C. A., Singh, H., Perry, A. S. & Prockop, D. J. The Wnt Signaling Inhibitor Dickkopf-1 Is Required for Reentry into the Cell Cycle of Human Adult Stem Cells from Bone Marrow. J. Biol. Chem. 278, 28067-28078 (2003).
18. Yu, H. et al. Effects of the Renin-Angiotensin System on the Current Ito in Epicardial and Endocardial Ventricular Myocytes From the Canine Hear. Circ Res 86, 1062-1068 (2000).

1. A method for regenerating myocardium in a mammal, comprising: delivering cells to the myocardium that induce native myocytes to enter the cell cycle.

2. The method according to claim 1, wherein the cells comprise at least one of stem cells, human stem cells and progenitor cells.

3. (canceled)

4. The method according to claim 1, wherein the mammal is a human.

5. (canceled)

6. The method according to claim 1, wherein the stem cells comprise at least one of human mesenchymal stem cells, human hematopoietic stem cells, human endothelial stem cells, and human embryonic stem cells.

7.-9. (canceled)

10. The method according to claim 1, wherein the cells are delivered via at least one of a scaffold, synthetic scaffold, biological scaffold, extra cellular matrix scaffold, an injection into the blood stream, an injection into a coronary artery, an injection into a coronary vein, an injection into the myocardium and an injection into the pericardial space.

11.-18. (canceled)

19. A method for regenerating myocardium in a mammal, comprising: attracting native stem cells to the myocardium that induce native myocytes to enter the cell cycle.

20. The method of claim 19, wherein the native stem cells are attracted to the myocardium by (i) excising a portion of the myocardium and (ii) replacing the excised portion with an extracellular matrix.

21. The method according to claim 19, wherein the stem cells comprise at least one of human stem cells and progenitor cells.

22. The method according to claim 19, wherein the mammal is a human.

23. (canceled)

24. The method according to claim 21, wherein the human stem cells comprise at least one of human mesenchymal stem cells, human hematopoietic stem cells, human endothelial stem cells, and human embryonic stem cells.

25.-27. (canceled)

28. The method according to claim 19, wherein stem cells are delivered via at least one of a scaffold, synthetic scaffold, biological scaffold, extra cellular matrix scaffold, an injection into the blood stream, an injection into a coronary artery, an injection into a coronary vein, an injection into the myocardium and an injection into the pericardial space.

29.-36. (canceled)

37. A solution that induces myocyte proliferation, comprising at least one of (i) media conditioned by stem cells and (ii) media conditioned by myocytes and stem cells when they are co-cultured together.

38. The solution according to claim 37, wherein the media conditioned by the stem cells and the media conditioned by the coculturing of the stem cells and myocytes are mixed together.

39. The solution according to claim 37, wherein the media conditioned by stem cells is used to incubate myocytes.

40. The solution according to claim 37, further comprising at least one of Wnt 5a, metalloproteases (MMPs), insulin-like growth factor, platelet derived growth factor and brain derived neurotrophic factor.

41.-47. (canceled)

48. A method for treating a subject afflicted with a cardiac disorder, in vivo, comprising (i) producing a solution comprising media conditioned from the culture of cells, in vitro, and (ii) administering the solution of step (i) to the subject, thereby treating the cardiac disorder in the subject.

49. The method of claim 48, wherein the cardiac disorder is at least one of a myocardial infarction, cardiomyopathy, congestive heart failure, ventricular septal defeet, a trial septal defect, congenital heart defeet, ventricular aneurysm, pediatric in origin, and a disorder requiring ventricular contruction.

50.-57. (canceled)

58. The method according to claim 48, wherein the cells comprise at least one of human stem cells, progenitor cells and stem cells.

59. The method according to claim 48, wherein the subject is a human.

60.-61. (canceled)

62. The method according to claim 58, wherein the human stem cells comprise at least one of human mesenchymal stem cells, human hematopoietic stem cells, human endothelial stem cells, and human embryonic stem cells.

63.-65. (canceled)

66. The method according to claim 48, wherein the solution is administered via at least one of a scaffold, synthetic scaffold, biological scaffold, extra cellular matrix scaffold, an injection into the blood stream, an injection into a coronary artery, an injection into a coronary vein, an injection into the myocardium and an injection into the pericardial space.

67.-136. (canceled)
